(19) United States
(12) Patent Application Publication
(10) Pub. No.: US 2005/0053655 A1
(43) Pub. Date: Mar. 10, 2005

(54) Rapid disintegrating tablets (RDTs) for pharmaceutical use and method for preparing the same

(75) Inventors: Chih-Chiang Yang, Taipei (TW); Wen-Che Wang, Taipei (TW); Hui-Yu Chen, Banchiau City (TW)

Correspondence Address: VENABLE, BAETJER, HOWARD AND CIVILETTI, LLP, P.O. BOX 34385, WASHINGTON, DC, 20043-9998, US

(73) Assignee: Pharmaceutical Industry Technology and Development Center, Wugu Shiang (TW)

(21) Appl. No.: 10/655,310

(22) Filed: Sep. 5, 2003

(51) Int. Cl. A61K 9/20; A61K 9/26; A61K 31/60

(52) U.S. Cl. 424/464; 514/165

(57) ABSTRACT

The present invention provides a fast-disintegrating tablet (RDT) and the method of preparing the RDT. The RDT contains a plurality of microcapsules which contains an active pharmaceutical ingredient surrounded by a polymeric matrix formed by a hydrogel. The microcapsules are separated from each other by a surfactant, particularly lecithin. The RDT is particularly suitable for use as a drug delivery system for antiacid or antiulcer drugs, such as famotidine. The RDT is further characterized by their its fast disintegration time of about 3 second to 3 minutes.

### FIELD OF THE INVENTION

This invention relates to a rapid disintegrating tablet (RDT) for pharmaceutical use. The RDT is characterized by its containing of a plurality of microcapsules/microspheres, each containing an active pharmaceutical ingredient surrounded with a polymeric matrix formed by cross-linking hydrogel. The microcapsules/microspheres are about 50 μm in diameter and have a rapid disintegrating time of about 3 seconds to 3 minutes, particularly between 10 seconds to 1 minute. The microcapsules/microspheres are further separated from each other by a surfactant, preferably lecithin, before compressed into a tablet. The RDT is particularly suitable for delivery antiacids/anti-ulcer agents, H₂-antagonists, anti-inflammatory agents, analgesics, and/or calcium channel blockers. This invention also relates to the method for making and using the RDT.

### BACKGROUND OF THE INVENTION

Rapid disintegrating tablets (RDTs) are often employed when the active ingredient is intended to act in a localized manner, rather than systemically. For example, antacids, non-steroidal anti-inflammatory drugs (NSAID), and/or analgesics are often administered in RDT form. RDTs can also be employed as an alternative to administering a number of smaller tablets when the active ingredient requires a relatively large dose in order to achieve the desirable therapeutic effect. A further reason for using RDTs is to enable the tablet to be reduced to a finely divided state quickly, thereby facilitating more rapid release and hence more rapid absorption of the active ingredients. RDTs can thus be useful for the treatment of conditions, such as gastroesophageal reflux disease (GERD), where a quick onset of action of the active ingredient is required. In that regard, histamine H₂-receptor antagonists, such as cimetidine, ranitidine, and/or famotidine, would be expected to be in the form of RDTs so as to be useful in the treatment of GERD.

Microencapsulation is a method for delivery of an active substance (such as an active pharmaceutical ingredient, enzymes, toxins, or other substances) by enveloping such substances in polymeric matrices. Over the years, microcapsulation have become an increasingly important technology in the development of both controlled release and taste masked pharmaceutical formulations.

Microencapsulation results in the formation of “microcapsules,” which are spherical aggregates with a diameter of about 0.1 to about 5 mm, each containing at least one solid or liquid core surrounded by at least one continuous membrane. More precisely, they are finely dispersed liquid or solid phases coated with film-forming polymers. Besides single-core microcapsules, there are also multiple-core aggregates, known as microspheres, which contain two or more cores distributed in the continuous membrane material. In addition, single-core or multiple-core microcapsules may be surrounded by an additional second, third etc. membrane.

Microcapsules have been widely used in controlled release or delayed release of drugs, but seldom in RDTs. The primary reason is because once the microcapsules are formed, the outer membrane (also called the coating layer) has been changed from primarily sticky material to a non-sticky insoluble matter, which is difficult to be disintegrated under such mild conditions as in a living body, and is difficult to quickly release its contents. Thus, although there have been many commercially available drugs in the market which contain microcapsules, such as Hallcrest Microcapsules, Coletica Thalaspheres, Lipotec Millicapseln, Induchem Unispheres, Unicerin C30, Kobo Glycospheres, Softspheres and Kuhs Probiol Nanospheres, none has been in RDTs.

Microencapsulation is generally carried out by suspending the active pharmaceutical ingredient in an aqueous medium containing so-called “hydrogel” material that can be reversibly gelled, forming the suspension into droplets (generally by stirring or homogenization), followed by hardening the droplets as discrete, shape-retaining, water insoluble microcapsules. A typical microcapsulation method is called “a coacervation or crosslinking” process, in which microcapsules can be prepared by (1) preparation of an adhesive coating layer by forming a coacervate phase containing the active substances and the hydrogel materials; (2) stabilizing (or hardening) of the coating layer by varying the temperature, pH, or agitation speed of the materials, and (3) recovering the microcapsules by drying.

Hydrogels have been described since 1956 (U.S. Pat. No. 2,976,576) and subsequently a large number of patents have been issued describing the synthesis and use of hydrogels. Hydrogels are used as polymeric, inert carriers for active substances. Particularly, they have the capability of slowly and controllably releasing the active substances, such as drugs (U.S. Pat. Nos. 3,574,826; 3,577,512; 3,551,556; 3,520,949; 3,576,760; 3,641,237; 3,660,563); agricultural chemicals (U.S. Pat. No. 3,576,760); or fragrances (U.S. Pat. Nos. 3,567,118; 3,697,643), into the surrounding environment.

Hydrogels may consist of natural, semisynthetic or synthetic materials. Natural hydrogel materials are, for example, gum arabic, agar agar, agarose, maltodextrins, alginic acid and salts thereof (such as sodium or calcium alginate), fats and fatty acids, cetyl alcohol, collagen, chitosan, lecithins, gelatin, albumin, shellac, polysaccharides (such as starch or dextran), polypeptides, protein hydrolyzates, sucrose and waxes. Semisynthetic hydrogel materials are, for example, chemically modified celluloses, more particularly cellulose esters and ethers (such as cellulose acetate, ethyl cellulose, hydroxypropyl cellulose, hydroxypropyl methyl cellulose and carboxymethyl cellulose), and starch derivatives, more particularly starch ethers and esters. Synthetic hydrogel materials are, for example, polymers, such as polyacrylates, polyamides, polyvinyl alcohol or polyvinyl pyrrolidone.

Typically, depending on the hydrogel materials used for formation of the microcapules, there are two common ways to prepare microcapules.

The first one involves ionically cross-linking the hydrogel material with a solution of multivalent cations to harden the hydrogel shell of the microcapules. Examples of the hydrogel materials that can be used to prepare this type of microcapsules include, without limitation, a group of water-soluble gum such as gelatin, acacia, sodium alginate, propylene glycol alginate, carrageenan, agar, tragacanth, and/or carboxymethylcellulose. Of these, alginate and carrageenan are the ones particularly of interest due to their unique capability of forming spherical beads with encapsulated material. Briefly, the microcapsules are formed by ionotropic gelling, i.e., the alginate is dropped down into a calcium solution and the carrageenan into a potassium solution. However, the resulting beads are stable only in the presence of ions (calcium and potassium, respectively).

More recently, there has been reported that the use of ultrasonic nozzles has offered a new way of making smaller microcapsules with very good control over the size of the droplets. One preferred method for making this type of microcapsules is taught by U.S. Pat. No. 4,352,883 to Lim. Using this method, 500 μm diameter (sized by gel filtration) capsules with a permeability of approximately 6,000 to 40,000 molecular weight are formed with a core of alginate cross-linked with calcium ions selectively coated with a poly-cationic skin using polymers such as poly-L-lysine and poly-vinylamine. The microcapsule size can be reduced (for example, to below 200 microns for injection) by increasing the spraying speed at the droplet forming stage. The process is as follows: the active substances are encapsulated in a physiologically-compatible medium containing a hydrogel material. The medium is then formed into droplets around the active substances and gelled by changing temperature, pH or ionic strength.

The second way to prepare microcapsules involves the use of known chemical crosslinking agents, such as formaldehyde or glutaraldehyde, to irreversibly crosslink the hydrogel material. Other crosslinking agents include tannic acid (tannin) or potassium aluminum sulfate (alum). An optional capsule hardening step, which applies particularly to pH or thermosensitive polymers, consists of adjusting the hydrogel material to various pH and/or temperatures.

Recently, there has been some reports relating to rapid disintegrating tablets for delivering antiacids/anti-ulcer drugs. For example, Japanese Patent No. 2002087958 discloses a fast disintegrating tablet containing famotidine, lactose with a 10% particle size of 30±20 μm, 50% particle size of 55±20 μm, and 90% particle size of 85±20 μm, a binder and a sweetener. The disintegration time of the tablet is 0.05-3 min. This fast disintegrating tablet, however, does not employ microencapsulation technologies.

In addition, WO 00/25754 describes an instant solid oral dosage form comprising famotidine and alginic acid, where both famotidine and alginic acid are used as therapeutic agents for treating gastric disorders. The two ingredients are physically separated by a barrier that is impermeable to alginic acid. This solid dosage form can be used as a chewable tablets, swallowable tablets, and hard and soft gel capsules, but not the RDTs.

In the present invention, a new type of rapid disintegrating tablets (RDT) will be described. The RDTs of the present invention utilize the microencapsulation technology to enclose an active pharmaceutical ingredient in a microcapsule or microsphere. The RDTs contain a plurality of microcapsules, each containing an active pharmaceutical ingredient surrounded by a hydrogel matrix, which is the polymeric matrix form of the hydrogel material. The microcapsules are separated from each other by a surfactant. Due to the rapid disintegrating time (3 second to 3 minutes) and small size of the microcapsules (about 50 μm in diameter), the RDT of the present invention is particularly useful for delivering drugs that cause irritation to the gastrointestinal tract, such as antiacids/anti-ulcer agents, H₂-antagonists, anti-inflammatory agents, analgesics, and calcium channel blockers, when introduced to the mucosa as a solid.

### SUMMARY OF THE INVENTION

The present invention provides a rapid disintegrating tablet (RDT) which contains a plurality of microcapsules and a disintegrant, each in the size of about 50 μm in diameter. The term “microcapsule,” as used hereinafter, is used interchangeable with microsphere, to denote small spherical beads containing at least one solid or liquid core surrounded by at least one continuous polymeric matrix, i.e., either single-core or multi-core aggregates.

The microcapsules contain an active pharmaceutical ingredient which is surrounded by a polymeric matrix formed by a hydrogel. The microcapsules are separated from each other by a surfactant and compressed into the RDT. The RDT is characterized by its fast disintegration time of about 3 second to 3 minutes, preferably between 10 seconds and 1 minute.

The active pharmaceutical ingredient which is suitable for use in the RDT includes antiacid or anti-ulcer agents (such as cimetidine, ranitidine, nizatidine, roxatidine, or famotidine); anti-inflammatory agents (such as indomethacin, ibuprofen, naproxen, prednisone, prednisolone, dexamethasone, or piroxicam); analgesics (such as aspirin); and calcium channel blockers (such nifedipine or amlodipine).

The hydrogel that is suitable for use in making the microcapsules includes, but is not limited to, gelatin, albumin, carboxymethylcellulose, polyvinyl alcohol, chitin, alginic acid or aginate. The preferred hydrogel is alginic acid or alginate (such as sodium alginate, potassium alginate, calcium alginate, and/or propylene glycol alginate). The alginic acid or alginate hydrogel polymeric matrix is formed by interacting the alginic acid or alginate with a calcium solution, such as CaCl₂ solution.

The surfactant that is used in segregating the microcapsules is preferred to be lecithin. The disintegrant is preferred to be Crospovione.

Optionally, the RDT contains an excipient which is starch, mannitol, lactose, sorbitol, polyethylene glycol (PEG) 6000, or a mixture thereof. Optionally, the RDT contains a flavor, a sweetener, and/or effervescent salts.

The present invention also provides a method for preparing the RDT, which includes (1) dispersing the active pharmaceutical ingredient in a hydrogel to form a microcapsule-pre-forming solution; (2) gelling or hardening the polymeric matrix of the hydrogel in the microcapsule-pre-forming solution to form microcapsules; (3) mixing a surfactant with the microcapsules to prevent aggregation of the microcapsules; (4) granulating the microcapsules to microcapsule granules; and (5) compressing the microcapsule granules into the RDT.

The preferred hydrogel for preparing the RDT is alginic acid or alginate. When alginic acid or alginate is used, it is preferred to spray the mixture of active pharmaceutical ingredient and alginic acid/alginate solution through a jet nozzle into a solution containing a CaCl₂ solution to form a microcapsule-containing solution. Using this method, the active pharmaceutical ingredient is within the polymeric matrix formed by ionically cross-linking alginic acid/alginate with Ca⁺². The microcapsules are collected by filtration through, for example, a funnel.

The preferred active pharmaceutical ingredient to be used in the RDT is an antiacid or anti-ulcer agent, particularly famotidine. Famotidine is preferred to be micronized prior to the mixing with the alginic acid or alginate.

Finally, the present invention provides a method for using the antiacid or antiulcer agent-containing RDT in treating patients with gastroesophageal reflux disease (GERD) or gastric diseases by orally administering the RDT containing the antiacid or antiulcer agent to the patients.

### BRIEF DESCRIPTION OF THE DRAWINGS

FIG. 1 shows a flowchart of the manufacturing process for making famotidine-containing microcapsule granules in which famotidine and alginate are microencapsulated within the microcapsules/microspheres.

FIG. 2 shows a flowchart of the manufacturing process for making the famotidine-containing rapid disintegrating tablet (RDT) from the famotidine-containing microcapsule granules of FIG. 1.

FIG. 3 shows the morphology of the famotidine-containing microspheres under microscope. The bar represents 50 μm.

FIG. 4 shows a time course of plasma concentrations between the RDTs of Lot FA13 (□) and commercially available product, Gaster® (♦) 20-mg tablet. Gaster® is a brand name product of famotidine sold in Japan.

FIG. 5 shows a time course of plasma famotidine concentrations in Formulation A (♦) and Formulation B (□) of the present invention. Both Formulations A and B have the same pharmaceutical composition as that of Lot FA09 except that the famotidine of Formulation A was microencapsulated and that of Formulation B was without microencapsulation.

### DETAILED DESCRIPTION OF THE INVENTION

Chewable tablets containing active pharmaceutical particles coated with a membrane are a well-known dosage form. They are intended to disintegrate in the mouth under the action of chewing and typically they are larger than tablets which are intended to be swallowed. The advantages of chewable tablets over dosage forms for swallowing include improved bioavailability through the immediate disintegration, patient convenience through the elimination of the need for water and patience acceptance through their pleasant taste.

Nevertheless, a common problem of chewable tablets is that chewing can cause a breakdown of the membrane that coats the active particles. Furthermore, the extent of mastication, which is associated with the length of time in which a drug remains in the mouth, plays an important role in determining the amount of taste masking. As a result, the drug's unpleasant taste and throat grittiness are often perceived by the patient.

To overcome such problems, other solid dosage forms known as fast dispersing or rapid disintegrating tablets (RDTs) have been developed. RDTs containing particles of active ingredient can be rapidly disintegrated into many coated cores of active ingredient due to the presence of one or more disintegrating agents. However, the presence of such coated cores of active ingredient tends to weaken the structure of the RDTs, which leads to poor friability values for the RDTs. Accordingly, the use of RDTs are limited due to their limited physical integrity as evidenced by their high friability, as compared to the conventional tablet forms. In fact, RDTs have previously been found to fracture or chip easily and therefore require careful packaging and handling prior to placing them in the mouth.

In addition to the disintegrating agents, most of the RDTs contain other excipients, such as swelling agents or thickening agents, which are capable of producing a viscous medium that facilitates the suspension of the solid particles, when the tablets disintegrate directly in the mouth or in a glass of water. As a result, the total weight of the RDTs can be rather high, and thus less acceptable to patients, especially when high dosage of the active ingredient is required.

The present invention provides a RDT which not only has the advantages of rapid disintegrating the active ingredient into the mouth and/or the stomach, but also avoids the pitfall of high friability, high weight, and high viscosity as in the traditional RDT. The RDT of the present invention maintains the active pharmaceutical ingredient in a packed mass prior to their ingestion and then rapidly disintegrates in the mouth or into the gastric fluid to permit the active pharmaceutical ingredient to rapidly and evenly disperse in the stomach.

The invention uses a plurality of microcapsules/microspheres containing the active pharmaceutical ingredient surrounded by a polymeric matrix formed by a hydrogel capable of being cross-linking. The microcapsules/microspheres are then mixed with a surfactant to prevent them from forming viscous aggregates. The surfactant-treated microcapsules/microspheres are then granulated and compressed into the RDTs.

Hydrogels have been widely employed in controlled-release dosage forms. Hydrogel is a colloidal gel in which water is the dispersion medium. It includes ingredients that are dispersible as colloidals or soluble in water, organic hydrogels, natural and synthetic gums, and inorganic hydrogels. Examples of hydrophilic colloids include, but are not limited to, silica, bentonite, tragacanth, pectin, alginate, methylcellulose, sodium carboxymethylcellulose and alumina. The hydrophilic colloids are frequently used in the pharmaceutical formulations as a binder, disintegrant, film coating agent, suspending agent, rate-controlling agent, and thickening agent. An additional advantage of hydrogels, which has received considerable attention, is that they may provide desirable protection of drugs from the potentially harsh environment in the vicinity of the release site.

The hydrogel that is suitable for use in the RDTs of the present invention includes, but is not limited to, alginic acid, alginate, gelatin, albumin, carboxymethylcellulose, polyvinyl alcohol, chitin, aminoalkyl methacrylate copolymer, carboxyvinyl polymer, polyvinyl acetal diethylamino acetate, carboxymethylethylcellulose, styrene maleic acid copolymer, sodium polyvinyl sulfonate, polyvinyl acetate, cellulose acetate butyrate, benzyl cellulose, ethyl cellulose, polyethylene, polystyrene, natural rubber, nitrocellulose, ketone resin, polymethyl methacrylate, polyamide resin, acrylonitrile-styrene copolymer, epoxy resin, vinylidene chloride-acrylonitrile copolymer, polyvinyl-formal, cellulose acetate, hydroxypropyl cellulose, hydroxypropylmethyl cellulose, hydroxymethylcellulose acetate succinate, vinyl chloridevinyl acetate copolymer, polyvinyl chloride, shellac, polyester, polycarbonate, cellulose acetate propionate, polyvinyl pyrrolidone, hydroxypropylmethyl cellulose phthalate, phenylsiloxane ladder polymer, polyacetic acid, polyglycolic acid, polylactic acid glycolic acid copolymer, polyglutamic acid and polylysine. Among these, alginic acid and/or aginate is the preferred hydrogel to be used in the RDTs.

Alginate is the salt or ester of alginic acid. Examples of alginate include, but are not limited to, sodium alginate, potassium alginate, propylene glycol alginate, and mixtures thereof. Alginic acid and alginate can be extracted from various species of brown algae (seaweed). Alginic acid is a high molecular weight linear polysaccharides consisting of a mixture of β-(1→4)-D-mannosyluronic acid (MA) and α-(1→4)-L-gulosyluronic acid (GA) residues. The percentages of MA and GA residues in the polysaccharide vary, depending on the specific species of algae used in the manufacturing. The characteristics of viscosity and reactivity of different alginates also depend on the specific source of algae and the ions in solution. The common alginates used in the pharmaceutical industries are sodium alginate, potassium alginate, and propylene glycol alginate. Alginate products in different grades are commercially available.

Alginate is generally acid stable and heat resistant. The monovalent alginate salt hydrates rapidly in cold or hot water. It is a film former. It reacts with multivalent cations (especially calcium) to form thermally irreversible gels. Adjusting the concentration of calcium ions, which cause cross-linking, controls the gel strength.

Therapeutically, high doses of alginic acid or alginate have been used in the treatment of gastrointestinal disorders. After ingestion, alginic acid or alginate forms a raft on the top of the contents of the stomach, which serves as a physical barrier to regurgitation and, in the event that reflux does occur, will preferentially bathe the esophageal mucosa, thereby protecting it from exposure to gastric contents.

The active pharmaceutical ingredient that is particularly suitable for the RDTs includes drugs that are acid-sensitive or of limited solubility in gastric fluid but are capable of acting locally within the gastrointestinal tract or systemically by absorption into circulation via the gastrointestinal mucosa. This include, but is not limited to antiacid/antiulcer agents (such as calcium carbonate, and H₂-antagonists [e.g., cimetidine, ranitidine, nizatidine, roxatidine or famotidine]); anti-arthritic or anti-inflammatory agents (such as indomethacin, ibuprofen, naproxen, prednisone, prednisolone, dexamethasone, and piroxicam); analgesics (such as aspirin), and/or calcium channel blockers (such as nifedipine and amlodipine). Among these, the pharmaceutically active ingredients with special anti-antacid action or having an acid-neutralizing capacity are the preferred ones for the RDTs.

In the Examples illustrated below, infra, a RDT using famotidine as the active pharmaceutical ingredient will be described. Famotidine is a white to pale yellow crystalline compound that is very slightly soluble in water. Famotidine is currently commercially available in both injectable and tablets under both brand name as well as generic names. The brand name famotidine is under the tradename of Pepcid® sold by Merck. Famotidine is particularly potent in treatments of duodenal ulcer, benign gastric ulcer, benign gastric ulcer, gastroesophageal reflux disease (GERD) and pathogenic hypersecretory conditions.

One of the problems associated with famotidine is that it contains two polymorphic forms, with form A being powder-like and from B being needle-like (which can be agglutinated into nodes), as described in U.S. Pat. No. 5,120,850. However, since most of the commercially available famotidine contains a mixture of the two polymorphic forms, it is preferred to micronize or mill the famotidine before mixing with alginic acid or alginate.

The present invention utilizes a surfactant to segregate the microcapsules/microspheres from each other so as to avoid forming viscous aggregates or colloids. Examples of the surfactant include, but are not limited to, any conventional surfactants used in the oral dosage forms that are familiar to those in the art. The preferred surfactant is lecithin. Lecithin helps in encapsulation and is a good dispersing agent. Lecithin from a soybean source is particularly favorable. Egg-derived lecithin is less favorable due to lower stability and possibility of viral or bacterial contamination.

The RDTs of the present invention also contains a disintegrant. The preferred disintegrant is Crospovione.

Other excipients, such as diluents and/or fillers, can also be added in the RDTs of the present invention. Examples of diluents/fillers include, but are not limited to, any conventional diluents/fillers commonly used in the oral dosage forms that are familiar to those in the art. The preferred diluents/fillers are starch and lactose.

The present invention also provides a method for preparation the RDTs. Briefly, the active pharmaceutical ingredient is first homogenized or thoroughly stirred with an aqueous solution of a hydrogel to form a microcapsule-pre-forming solution. The hydrogel is then gelled or hardened by varying methods based on the property of the hydrogel. For example, if the hydrogel is alginic acid or alginate, the microcapsule-pre-forming solution is passing through a jet nozzel into a CaCl₂ solution. A cross-linking between the alginic acid/alginate and the Ca⁺² ion is formed which becomes the outer membrane for the active pharmaceutical ingredient.

Other gelling or hardening methods include, for example, in the use of synthetic polymer or co-polymer as the hydrogel, the use of a “coacervation or crosslinking” process, which involves the use of crosslinking agents, such as formaldehyde, glutaraldehyde, tannic acid, or potassium aluminum sulfate to irreversibly crosslink the hydrogel material. In addition, if the Eurigit® series of polymer or co-polymer (such as polymethyl methacrylate) is used, the hardening hardening step also applies to change of pH or temperatures.

The microcapsule-containing solution is further filtered and the microcapsules. The filtered microcapsules are mixed with the surfactant and optionally diluents/fillers, and wet granulated to form microcapsules granules. The resultant granules are dried and sieved and the content of the active pharmaceutical ingredient is analyzed. The granules are further blended with other pharmaceutical acceptable excipients, including optionally, but are not limited to, a disintegrant, a sweetener, a flavor, a lubricant, and an effervescent. The mixture is then sieved and compressed into tablets.

The following examples are illustrative, but not limiting the scope of the present invention. Reasonable variations, such as those occur to reasonable artisan, can be made herein without departing from the scope of the present invention. Also in describing the invention, specific terminology is employed for the sake of clarity. However, the invention is not intended to be limited to the specific terminology so selected. It is to be understood that each specific element includes all technical equivalents which operate in a similar manner to accomplish a similar purpose.

### EXAMPLE 1

### Composition, Preparation and Test Results of Famotidine RDT, Lot Fa08

Composition and Preparation

The formulations of three (3) Lots of famotidine-containing RDTs (i.e., FA08, FA09, and FA13) are listed in Table 1.

TABLE 1
Percentages of Ingredients in 3 Lots
of Famotidine-Containing RDTs

| | | | |
|---|---|---|---|
| Ingredients | FA08 | FA09 | FA13 |

Microcapsules/Microspheres (% of total by weight)

| | | | |
|---|---|---|---|
| Famotidine | 6* | 6* | 6* |
| Alginate | 1.5* | 1.5* | 1.5* |

Surfactant (% of total by weight)

| | | | |
|---|---|---|---|
| Tween 60 | 0.9 | 0 | 0 |
| Lecithin | 0 | 0.9 | 0 |

Excipients (% of total by weight)

| | | | |
|---|---|---|---|
| Starch | 20 | 20 | 20.5 |
| Lactose | 14.6 | 14.6 | 15 |
| Mannitol | 37 | 37 | 37 |
| Sorbitol | 5 | 5 | 5 |
| PEG 6000 | 5 | 5 | 5 |
| Crospovidone | 10 | 10 | 10 |

Additives (% of total by weight)

| | | | |
|---|---|---|---|
| Orange flavor | 0.5 | 0.5 | 0.5 |
| Aspartame | 0.5 | 0.5 | 0.5 |
| Mg stearate | 0.5 | 0.5 | 0.5 |
| Aerosil 200 | 0 | 0 | 0.5 |
| Citric Acid:NaHCO₃** | 5 | 5 | 5 |
| (5:8) | | | |

*Active ingredients were first mixed with alginate, then passed through a 0.25 M CaCl₂ solution to form microcapsules/microspheres.
**Citric acid and NaHCO₃ are used as effervescent.

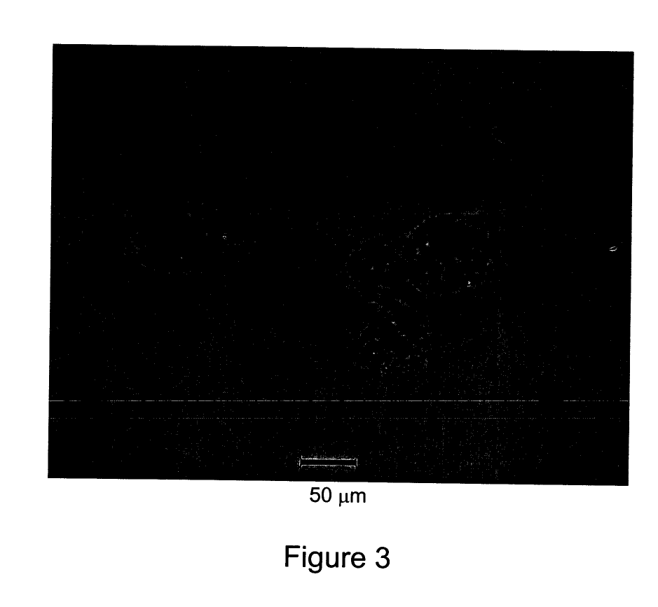

After micronization or milling, about 20 g of the milled famotidine was dispersed in 1000 mL of 1% alginate. The mixture was then passed through a jet nozzle into a 0.25 M CaCl₂ solution to form famotidine-containing microcapsules/microspheres. The famotidine-containing microcapsules/microspheres were spherical particles with about 50 μm in diamter, as shown in FIG. 3.

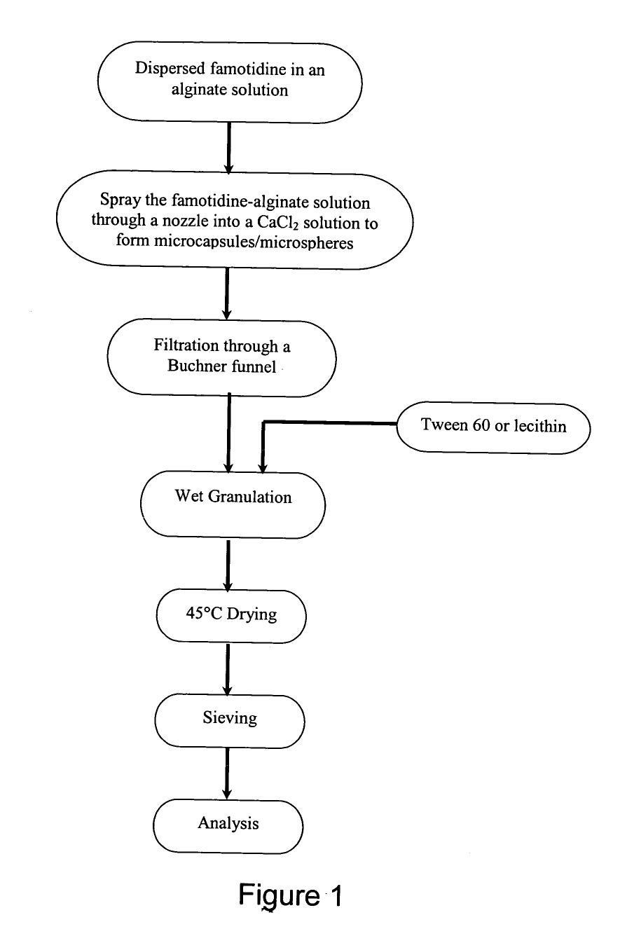

The microcapusles/microspheres were collected by filtration through, for example, a Buchner funnel. The collected microcapsules/microspheres were further mixed with starch and lactose in the amounts corresponding to the ratios listed in Table 1. In FA08, 0.9% of Tween 60 was added to the mixture of microcapsules/microspheres and starch/lactose. In FA09, 0.9% of lecithin were added. In FA13, no Tween 60 or lecithin were added. The mixture was then wet granulated to form wet granules. The wet granules were then dried at 45° C., sieved and then analyzed for composition. A flow-chart of the preparation of RDTs according to the present invention is depicted in FIG. 1.

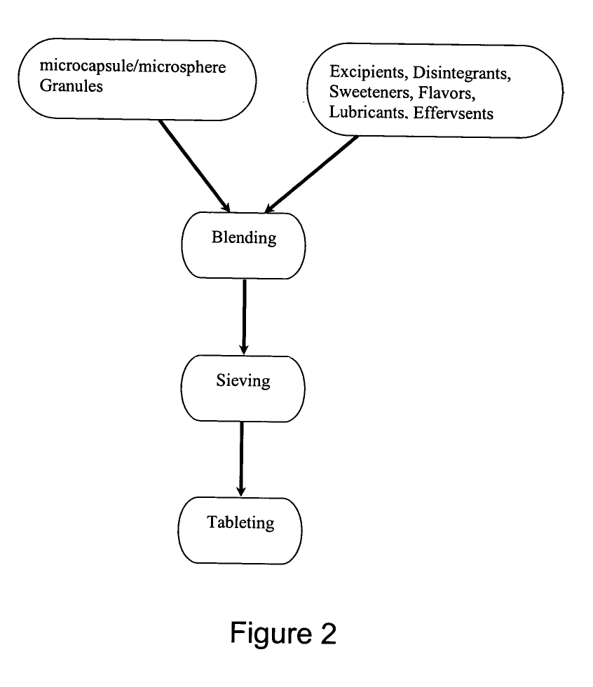

The granules were blended with mannitol, sorbitol, PEG 6000, Crospovidone, orange (flavoring agent), aspartame (sweetness), magnesium stearate (lubricant), citric acid and NaHCO₃ (effervesent salts) in the amounts corresponding to the percentages listed in Table 1. The resultant blend was sieved and then compressed into tablets. A flowchart depicting the manufacturing process of making the tablets from the microcapsules/microspheres-containing granules to the RDT is provided in FIG. 2.

Test Methods and Results

Stability testing: Sufficient quantities of famotidine RDTs (Lot FA08) were separately placed in stability chambers under 25° C. and 60% relative humidity (RH); 30° C. and 60% RH; or 40° C. and 75% RH. After 1, 2 and 3 months of storage, RDT samples were selected from each of the three stability chambers. The RDT samples were tested for famotidine content (%), tablet weight (mg), hardness (Newton), disintegration time (seconds), and friability (%) using conventional test methods, such as those listed in U.S. Pharmacopoeia.

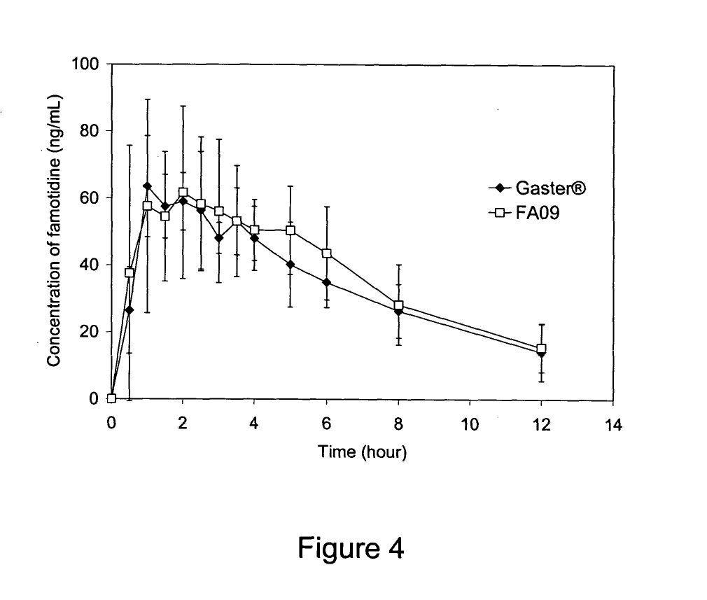

The testing results of famotidine RDT Lot FA08 after manufacturing (0 month) and after storage in stability chambers after 1, 2, and 3 months are listed in Table 2. The contents of famotidine in the RDT Lot FA08 over time under various storage conditions are shown in FIG. 4.

TABLE 2
Stability Test Results of Famotidine RDT in Lot. FA08

| | | Famotidine | | Hardness | disintegration | |
|---|---|---|---|---|---|---|
| Storage | Storage | content (%) | Tablet weight | (Newton) | time (sec) | Friability |
| time | condition | (n = 6) | (mg) | (n = 6) | (n = 5) | (%) |
| 0 month | | 100.0 | 373.2 ± 2.7 | ND | 55 ± 12 | 28.4 |
| 1 month | 25° C., 60% RH | 98.8 | 374.8 ± 2.0 | ND | 43 ± 12 | 5.8 |
| | 30° C., 60% RH | 94.9 | 373.0 ± 2.6 | ND | 38 ± 6 | 3.9 |
| | 40° C., 75% RH | 88.2 | 374.0 ± 2.3 | ND | 27 ± 3 | 3.4 |
| 2 month | 25° C., 60% RH | 91.5 | 375.2 ± 3.7 | ND | 44 ± 6 | 5.0 |
| | 30° C., 60% RH | 85.5 | 375.0 ± 2.9 | 13.1 ± 0.4 | 38 ± 9 | 4.2 |
| | 40° C., 75% RH | 71.5 | 373.3 ± 2.7 | 14.5 ± 1.1 | 32 ± 7 | 4.3 |
| 3 month | 25° C., 60% RH | 83.8 | 373.4 ± 2.4 | ND | 42 ± 10 | 7.3 |
| | 30° C., 60% RH | 78.5 | 374.2 ± 2.5 | 13.9 ± 0.7 | 44 ± 5 | 5.2 |
| | 40° C., 75% RH | 66.8 | 373.7 ± 3.3 | 13.5 ± 0.7 | 31 ± 6 | 6.6 |

n: numbers of samples tested
ND: not detectable
RH: relative humidity

### EXAMPLE 2

### Composition, Preparation and Test Results of Famotidine RDT, Lot Fa09

Composition and Preparation

The pharmaceutical composition of the famotidine RDT in Lot FA09 was provided in Table 1, supra.

The famotidine RDT was first micronized or milled. Then, about 20 g of the milled famotidine was dispersed in 1000 mL of 1% alginate to form a microcapsules/microspheres mixture. This mixture was then passed through a jet nozzle into a 0.25 M CaCl₂ solution to form the famotidine-alginate microcapsules/microspheres. The microcapsules/microspheres were then collected from the CaCl₂ solution by filtration through, for example, a Buchner funnel. The collected microcapsules/microspheres were further mixed with starch, lactose, and lecithin in the amounts corresponding to the percentages listed in Table 1, and then wet granulated. The granules were dried at 45° C., sieved and then the chemical composition of the granules was analyzed.

The granules were further blended with mannitol, sorbitol, PEG 6000, Crospovidone, orange flavor, aspartame, magnesium stearate, citric acid and NaHCO₃ in the amounts corresponding to the ratios listed in Table 1, supra. The resultant blend was sieved and then compressed into tablets.

Test Methods and Results

Stability testing: Sufficient quantities of famotidine RDTs (Lot FA09) were separately placed in stability chambers under 25° C. and 60% relative humidity (RH); 30° C. and 60% RH; or 40° C. and 75% RH. After 1, 2 and 3 months of storage, the famotidine RDT samples were selected from each of the three stability chambers and tested for famotidine content (%), tablet weight (mg), hardness (Newton), disintegration time (seconds), and friability (%) using conventional test methods, such as those listed in the U.S. Pharmacopoeia.

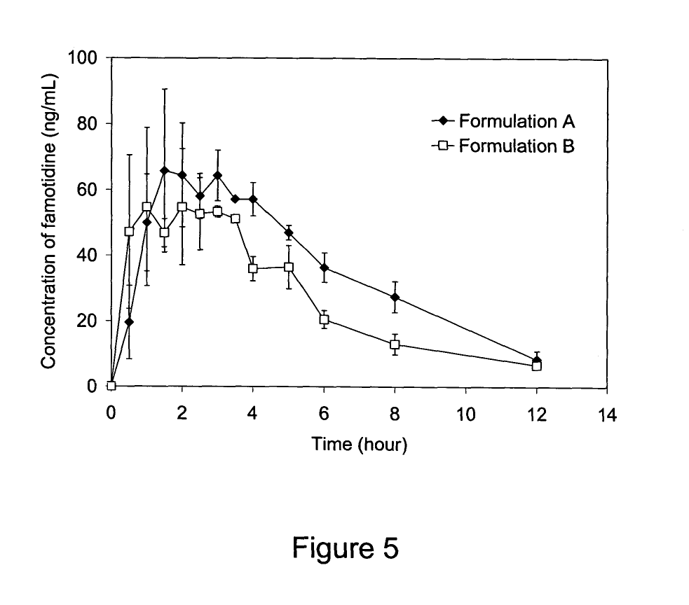

The testing results of famotidine RDTs in Lot FA09 after manufacturing (0 month) and after storage in stability chambers for 1, 2, and 3 months are listed in Table 3. The contents of famotidine RDTs in Lot FA09 over time under various storage conditions are also shown in FIG. 5.

TABLE 3
Stability test Results of Famotidine RDTs In Lot. FA09

| | | famotidine | | hardness | disintegration | |
|---|---|---|---|---|---|---|
| Storage | Storage | content (%) | Tablet weight | (Newton) | time (sec) | Friability |
| time | condition | (n = 6) | (mg) | (n = 6) | (n = 5) | (%) |
| 0 month | | 100.0 | 323.4 ± 2.6 | ND | 29 ± 4 | 100 |
| 1 month | 25° C., 60% RH | 101.2 | 323.1 ± 2.3 | ND | 26 ± 7 | 100 |
| | 30° C., 60% RH | 100.9 | 323.8 ± 3.6 | ND | 24 ± 4 | 100 |
| | 40° C., 75% RH | 95.9 | 323.2 ± 3.5 | ND | 25 ± 4 | 100 |
| 2 month | 25° C., 60% RH | 94.9 | 325.3 ± 4.1 | ND | 27 ± 5 | 100 |
| | 30° C., 60% RH | 95.4 | 323.1 ± 4.0 | ND | 25 ± 5 | 100 |
| | 40° C., 75% RH | 95.6 | 322.4 ± 3.7 | ND | 24 ± 5 | 100 |
| 3 month | 25° C., 60% RH | 96.0 | 323.0 ± 4.6 | ND | 30 ± 4 | 100 |
| | 30° C., 60% RH | 96.9 | 326.9 ± 7.0 | ND | 32 ± 11 | 100 |
| | 40° C., 75% RH | 94.0 | 322.2 ± 6.2 | ND | 36 ± 4 | 100 |

n: numbers of samples tested
ND: not detectable
RH: relative humidity

### EXAMPLE 3

### Composition, Preparation and Test Results of Famotidine RDTs in Lot Fa13

Composition and Preparation

The pharmaceutical composition of famotidine RDT in Lot FA13 was listed in Table 1, supra.

The famotidine RDT was first micronized or milled. Then, about 20 g of the milled famotidine was dispersed in 1000 mL of 1% alginate to form a microcapsules/microspheres mixture. This mixture was then passed through a jet nozzle into a 0.25 M CaCl₂ solution to form the famotidine-alginate microcapsules/microspheres. The microcapsules/microspheres were then collected from the CaCl₂ solution by filtration through, for example, a Buchner funnel. The collected microcapsules/microspheres were further mixed with starch and lactose (no surfactant, such as Tween 60 or lecithin, was added to the mixture) in the amounts corresponding to the percentages listed in Table 1, and then the microcapsules/microspheres and starch/lactose mixture was wet granulated. The granules were dried at 45° C., sieved and then the chemical composition of the granules was analyzed.

The granules were further blended with mannitol, sorbitol, PEG 6000, Crospovidone, orange flavor, aspartame, magnesium stearate, citric acid and NaHCO₃ in the amounts corresponding to the ratios listed in Table 1, supra. The resultant blend was sieved and then compressed into tablets.

Test Methods and Results

Stability testing: Sufficient quantities of famotidine RDTs (Lot FA13) were separately placed in stability chambers under 25° C. and 60% relative humidity (RH); 30° C. and 60% RH; or 40° C. and 75% RH. After 1, 2 and 3 months of storage, samples were selected from tablets in each of the three stability chambers. The samples were tested for famotidine content (%), tablet weight (mg), hardness (Newton), disintegration time (seconds), and friability (%) using conventional test methods, such as those listed in the U.S. Pharmacopoeia.

The testing results of famotidine RDTs in Lot FA13 after manufacturing (0 month) and after storage in stability chambers after 1, 2, and 3 months are listed in Table 4. The contents of the famotidine RDTs in Lot FA13 over time under various storage conditions are presented in FIG. 6.

TABLE 4
Stability Test Results of Famotidine RDTs in Lot. FA13

| Storage | Storage | famotidine | Tablet weight | hardness | disintegration | Friability |
|---|---|---|---|---|---|---|
| time | condition | content (%) | (mg) | (Newton) | time (sec) | (%) |
| 0 month | | 100.0 | 367.1 ± 2.5 | 24.2 ± 1.4 | 32 ± 2 | 1.3 |
| 1 month | 25° C., 60% RH | 95.8 | 367.8 ± 2.9 | 27.9 ± 2.5 | 29 ± 6 | 1.1 |
| | 30° C., 60% RH | 92.7 | 367.6 ± 2.9 | 30.1 ± 2.5 | 33 ± 3 | 0.8 |
| | 40° C., 75% RH | 84.0 | 366.8 ± 2.7 | 18.3 ± 0.5 | 24 ± 1 | 1.3 |
| 2 month | 25° C., 60% RH | 101.8 | 365.8 ± 4.2 | 23.9 ± 1.5 | 34 ± 5 | 0.8 |
| | 30° C., 60% RH | 93.2 | 3663 ± 3.6 | 26.7 ± 2.2 | 30 ± 2 | 0.9 |
| | 40° C., 75% RH | 80.7 | 367.0 ± 2.8 | 13.6 ± 0.8 | 23 ± 3 | 2.3 |
| 3 month | 25° C., 60% RH | 93.4 | 366.6 ± 2.9 | 30.0 ± 2.1 | 40 ± 8 | 0.2 |
| | 30° C., 60% RH | 87.9 | 365.2 ± 4.2 | 24.1 ± 2.8 | 46 ± 7 | 1.1 |
| | 40° C., 75% RH | 81.6 | 366.2 ± 3.2 | 10.4 ± 1.4 | 51 ± 12 | 3.4 |

N: numbers of samples tested
ND: not detectable
RH: relative humidity

Summary of Stability Studies from Lots FA08. FA09, and FA13:

The results from the stability studies of Lots FA08, FA09, and FA13 demonstrates as follows:

(1) With regard to the test of the famotidine content in the RDTs of Lots FA08, FA09, and FA13 after 0, 1, 2, and 3 months of storage, and after stored at 25° C., 60% relative humidity, 30° C., 60% relative humidity, and 40° C., 60% relative humidity, the famotidine content in Lot FA09 appeared to achieve the highest retention rate (% of original) of famotidine (between 94% and 101% in various conditions), contrasting to those in Lot FA08 (betweem 66% and 98.8%) and in Lot FA13 (between 81.6% to 95.8%).

(2) With regard to the weight of the tablet (mg), there appeared to be no significance difference in retaining the total weight of the RDT among the Lots, all showing almost no loss in the total weight of the tablets.

(3) With regard to hardness, the RDTs of Lot FA09 showed no detectable hardness under Newton test. Some of the RDTs of Lot FA08 (i.e., at 25° C., 30° C., and 40° C., and 60% RH and 75% RH at up to 1 month of storage) also did not show any detectable hardness. However, some other RDTs of Lot FA08, after being stored at 30° C. in 60% RH and 40° C. in 75% RH, and after 2 months of storage, began to show hardness in the range of 13.1 to 13.9 Newton. As for the RDTs of Lot FA13, all have shown detectable hardness, ranging from 10.4 to 30.1.

(4) With regard to disintegration time, the RDTs of Lot FA09 demonstrated the shortest disintegration with an average of 29+4 seconds of disintegration time. The RDTs of Lot FA13 was a short distance behind, with an average of 32±2 seconds of disintegration time. The RDTs of Lot FA08 had an average disintegration time of 55±12 seconds.

(5) With regard to friability (%), the RDTs of Lot FA09 had a percentage of relative friability (%) of 100%, contrasting to those of Lot FA08 (with friability ranging between 3.4% and 28.4%) and those of Lot FA13 (with friability ranging between 0.2% and 3.4%).

Thus, in sum, among the three different Lots of famotidine RDTs tested, the RDTs from Lot FA09 clearly showed the best stability results in famotidine content retention, the least hardness in all storage conditions, the lowest disintegration time, and the higest friability. The famotidine RDTs from Lot FA08 were superior to those from Lot FA13 in terms of the degree of hardness and % of friability but were inferior to those from Lot FA13 in terms of the famotidine content retention and disintegration time. The tablet weight retention among the RDTs of Lots FA08, FA09, and FA13 were about the same.

Because the only difference among the RDTs of Lots FA08, FA09, and FA13 was that the RDTs of Lot FA09 contained surfactant lecithin, the RDTs of Lot FA08 contained surfactant Tween 60, and the RDTs of Lot FA13 contained neither lecithin nor Tween 60, it was therefore concluded that lecithin improved the stability of the RDTs.

### EXAMPLE 4

### Pharmacokinetics of Famotidine RDTs

Materials and Method

A comparative study between the pharmacokinetics of the famotidine RDTs from Lot FA09 and a commercially available famotidine tablet Gaster® was conducted in humans. The study was conducted by collecting blood samples from four (4) healthy human subjects at 0, 0.5, 1, 1.5, 2, 2.5, 3, 3.5, 4, 5, 6, 8, and 12 hours after oral adminstration of the famotidine tablets. For each subjects, one dose of the reference drug (Gaster® 20 mg tablet) or one dose of the test drug (also in 20 mg tablet of famotidine RDTs from Lot FA09) was given. Famotidine concentrations in the plasma of the blood samples were analyzed using a conventional quantification analytical method, such as those listed in the U.S. Pharmacopoeias and well known to people skilled in the art, or High Performance Liquid Chromotography (HPLC).

Results

The average (mean±standard deviation (SD)) plasma concentrations of famotidine over time are presented in FIG. 4 and the average values of the pharmacokinetic parameters are presented in Table 5.

TABLE 5
Pharmacokinetic parameters (mean ± SD) of the reference
drug (Gaster ® famotidine 20 mg tablet) and the test
drug (famotidine RDTs of Lot FA09)

| | | Famotidine RDT |
|---|---|---|
| Pharmacokinetic | Gaster ® | of Lot FA09 |
| Parameters | mean ± SD (% cv) | Mean ± SD (% cv) |
| Cₘₐₓ (ng/mL) | 71.2 ± 11.8 (16.6) | 67.4 ± 24.7 (36.7) |
| Tₘₐₓ (hr) | 1.38 ± 0.75 (54.5) | 2.13 ± 1.93 (90.9) |
| AUC₀₋ₜ (hr · ng/mL) | 418.1 ± 89.1 (21.3) | 459.2 ± 142.7 (31.1) |
| AUC_{0-∞} (hr · ng/mL) | 533.2 ± 209.1 (39.2) | 552.8 ± 191.0 (34.6) |
| AUC ratio | 82.0 ± 12.1 (14.8) | 83.9 ± 5.2 (6.2) |
| T_{1/2} (hr) | 6.75 ± 3.14 (46.5) | 5.76 ± 0.87 (15.1) |

Cₘₐₓ: maximum plasma concentration
Tₘₐₓ: time to reach the maximum plasma concentration
AUC₀₋ₜ: area under the curve from time 0 to the time of the last measurable concentration
AUC_{0-∞}: area under the curve from time 0 and extrapolated to infinity
AUC ratio: the ratio of AUC₀₋ₜ/AUC_{0-∞}
T_{1/2}: terminal elimination half-life

Although there appeared to be large variations among subjects being tested with the reference drug (Gaster®) and the test drug (famotidine RDTs from Lot FA09), the pharmacokinetics parameters of the famotidine RDTs from Lot FA09 appeared to be bioequivalent to and comparable to those of the reference drug.

### EXAMPLE 5

### Pharmacokinetics Between Formulation A And Formulation B

Materials and Method

To study the pharmacokinetics of the famotidine RDTs between formulation A (i.e., RDTs from Lot FA09) and formulation B (i.e., RDTs from Lot FA13), blood samples were collected from two (2) groups of healthy humans subjects at 0, 0.5, 1, 1.5, 2, 2.5, 3, 3.5, 4, 5, 6, 8, and 12 hours. One dose of Formulation A (famotidine RDTs with microencapsulation) or one dose of Formulation B (famotidine RDTs without microencapsulation) was given to each of these human subjects. The plasma famotidine concentrations in plasma of the human subjects were determined using a conventional quantification analytical method, such as those listed in the U.S. Pharmacopoeias and well known to people skilled in the art, or High Performance Liquid Chromatograph (HPLC).

Results

The plasma famotidine concentrations of formulations A and B over time are shown in FIG. 5 and the average values of the pharmacokinetic parameters are shown in Table 6.

TABLE 6
Pharmacokinetic parameters (mean ± SD) of Formulation A
(with microencapsulation) and Formulation B (without
microencapsulation) in two Groups of Healthy subjects

| Pharmacokinetic | Formulation A | Formulation B |
|---|---|---|
| Parameters | (n = 4) | (n = 4) |
| Cₘₐₓ (ng/mL) | 73.2 ± 14.4 (19.7) | 62.1 ± 13.8 (22.3) |
| Tₘₐₓ (hr) | 2.0 ± 0.71 (35.4) | 2.0 ± 1.41 (70.7) |
| AUC₀₋ₜ (hr · ng/mL) | 433.8 ± 42.5 (9.8) | 310.0 ± 20.1 (6.5) |
| AUC_{0-∞} (hr · ng/mL) | 468.0 ± 56.6 (12.1) | 338.8 ± 14.7 (4.3) |
| AUC ratio | 92.8 ± 2.1 (2.3) | 91.5 ± 2.0 (2.1) |
| T_{1/2} (hr) | 3.97 ± 0.47 (12.0) | 3.59 ± 1.82 (50.7) |

Cₘₐₓ: maximum plasma concentration
Tₘₐₓ: time to reach the maximum plasma concentration
AUC₀₋ₜ: area under the curve from time 0 to the time of the last measurable concentration
AUC_{0-∞}: area under the curve from time 0 and extrapolated to infinity
AUC ratio: the ratio of AUC₀₋ₜ/AUC_{0-∞}
T_{1/2}: terminal elimination half-life

As shown in both FIG. 5 and Table 6, The Cₘₐₓ and AUC values of Formulation A were higher than those of Formulation B. The results suggested that microencapsulation of famotidine in the RDTs of the present invention increased the amounts of famotidine absorbed in comparison to a similar formulation without microencapsulating famotidine.

While the invention has been described by way of examples and in term of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

1. A rapid disintegrating tablet (RDT) comprising a plurality of microcapsules;
said microcapsules comprising an active pharmaceutical ingredient within a polymeric matrix formed by a hydrogel;
wherein said microcapsules are separated from each other by a surfactant; and
wherein said RDT has a disintegration time of about 3 second to 3 minutes.

2. The RDT according to claim 1, wherein said microcapsules are about 50 μm in diameter.

3. The RDT according to claim 1, wherein said RDT has a disintegration time of about 10 seconds to 1 minute.

4. The RDT according to claim 1, wherein said active pharmaceutical ingredient is an antiacid or anti-ulcer agent.

5. The RDT according to claim 4, wherein said antiacid or antiulcer agent is cimetidine, ranitidine, nizatidine, roxatidine, or famotidine.

6. The RDT according to claim 4, wherein said antiacid or antiulcer agent is famotidine.

7. The RDT according to claim 1, wherein said active pharmaceutical ingredient is an anti-inflammatory agent.

8. The RDT according to claim 7, wherein said anti-inflammatory agent is indomethacin, ibuprofen, naproxen, prednisone, prednisolone, dexamethasone, or piroxicam.

9. The RDT according to claim 1, wherein said active pharmaceutical ingredient is an analgesic.

10. The RDT according to claim 9, wherein said analgesic is aspirin.

11. The RDT according to claim 1, wherein said active pharmaceutical ingredient is a calcium channel blocker.

12. The RDT according to claim 11, wherein said calcium channel blocker is nifedipine or amlodipine.

13. The RDT according to claim 1, wherein said hydrogel is a hydrophilic polymer which is at least one selected from the group consisting of gelatin, albumin, carboxymethylcellulose, polyvinyl alcohol, and chitin.

14. The RDT according to claim 1, wherein said hydrogel is alginic acid or alginate.

15. The RDT according to claim 14, wherein said alginate is sodium alginate, potassium alginate, calcium alginate, propylene glycol alginate or mixtures thereof.

16. The RDT according to claim 14, wherein said polymeric matrix of said alginic acid or alginate is formed by interacting said alginic acid or alginate with a calcium solution.

17. The RDT according to claim 16, wherein said calcium solution is a CaCl₂ solution.

18. The RDT according to claim 1, wherein said surfactant is lecithin.

19. The RDT according to claim 1, further comprising an excipient which is at least one selected from the group consisting of starch, mannitol, lactose, sorbitol, and polyethylene glycol (PEG) 6000.

20. The RDT according to claim 1, further comprising a disintegrant, wherein said disintegrant is Crospovione.

21. The RDT according to claim 1, further comprising a flavor, a sweetener, and/or effervescent salts.

22. A method for preparing the rapid disintegrating tablet (RDT) according to claim 1 comprising:
dispersing the active pharmaceutical ingredient in a hydrogel to form a microcapsule-pre-forming solution;
gelling or hardening said microcapsule-pre-forming solution to form microcapsules;
mixing a surfactant with said microcapsules to form a surfactant-microcapsules mixture;
granulating said surfactant-microcapsules mixture to form microcapsule granules;
compressing said microcapsule granules into said RDT.

23. The method according to claim 22, wherein said hydrogel is alginic acid or alginate.

24. The method according to claim 22, wherein said microcapsules are formed by
spraying said microcapsule-pre-forming solution through a jet nozzle into a CaCl₂ solution to form a microcapsule-containing solution, wherein said active pharmaceutical ingredient is within said polymeric matrix formed by alginate; and wherein said microcapsules are collected by filtering said microcapsule-containing solution.

25. The method according to claim 22, wherein said active pharmaceutical ingredient is an antiacid or anti-ulcer agent.

26. The method according to claim 25, wherein said antiacid or anti-ulcer agent is famotidine.

27. The method according to claim 26, wherein said famotidine is micronized.

28. The method according to claim 22, wherein said surfactant is lecithin.

29. A method for treating a patient suffered from gastroesophageal reflux disease (GERD) comprising orally administering said RDT according to claim 4 to said patient with GERD.

30. A method for treating a patient with gastric disorder comprising orally administering said RDT according to claim 4 to said patient with gastric disorder.
